(19) United States
(12) Patent Application Publication
(10) Pub. No.: US 2021/0032285 A1
(43) Pub. Date: Feb. 4, 2021

(54) METHODS, COMPOSITIONS, AND KITS FOR TRAPPING MODIFIED BIOMOLECULES

(71) Applicant: CELL IDX, INC., San Diego, CA (US)

(72) Inventor: David A. SCHWARTZ, Encinitas, CA (US)

(21) Appl. No.: 16/815,565

(22) Filed: Mar. 11, 2020

Related U.S. Application Data
(60) Provisional application No. 62/816,797, filed on Mar. 11, 2019.

(51) Int. Cl. C07K 1/22; C07K 1/34; C07K 1/36; G01N 33/58

(52) U.S. Cl. CPC C07K 1/22; G01N 33/58; C07K 1/36; C07K 1/34

(57) ABSTRACT

The present disclosure provides methods, compositions, and kits for trapping functionalized biomolecules. The methods, compositions, and kits utilize a trapping material comprising a reactive carbonyl group. The trapping material is designed to react rapidly and efficiently with a functionalized biomolecule that comprises a reactive amino group, thus removing the functionalized biomolecule from solution. The trapping material is therefore effective in the purification of target molecules after labeling reactions with an excess of the functionalized biomolecule.

### CROSS-REFERENCE TO RELATED APPLICATIONS

This application claims the benefit of U.S. Provisional Application No. 62/816,797, filed on Mar. 11, 2019, the disclosure of which is incorporated herein by reference in its entirety.

### BACKGROUND OF THE INVENTION

In modern bioanalytical and biochemical systems and analytical techniques, synthetic and naturally-occurring biomolecules are frequently modified by chemical treatment with a variety of reactive agents for a variety of purposes. In some cases, the biomolecules, for example polypeptides, polynucleotides, or polysaccharides, are modified to include a reactive moiety, so that the modified biomolecule can then be used in a conjugation reaction with other target molecules. Such conjugation methods have been used, for example, to attach various biomolecules to radioactive and nonradioactive metal chelates, to drugs, to antigens, and to reactive surfaces. The approaches have, in particular, facilitated the development of in vitro and in vivo diagnostic assays, as well as the development of effective therapeutic agents. Exemplary reagents, labeling methods, and uses are disclosed in PCT International Publication Nos. WO 01/70685 A2; WO 02/10431 A2; WO 02/10432 A2; WO 02/57422 A2; WO 2008/140452 A1; WO 2011/100493 A1; WO 2012/071428 A2; WO 2013/177046 A1; WO 2016/127149 A2; and WO 2018/017606 A1; and U.S. Patent Application Publication No. 2008/0221343 A1. See also Greg T. Hermanson, Bioconjugate Techniques, Academic Press.

One of the challenges in applying the above bioconjugation methods to the labeling of biomolecules relates to the separation of unreacted bioconjugates and their target molecules from one another following completion of the bioconjugation reaction. Even with highly selective and highly efficient bioconjugation reactions, such as those described in the above publications, there is typically at least one residual, unreacted component of the reaction present in the reaction solution after completion of a given bioconjugation reaction. This is particularly true when an excess of the reactive agent, for example an excess of a functionalized biomolecule, is used in the reaction in order to maximize the extent of modification of the target molecule. Such conditions are often necessary where the target molecule, for example a target antibody molecule or a target oligonucleotide, is particularly valuable, or is available only in short supply. Use of an excess of the functionalized biomolecule or other reactive agent is then desirable in order to maximize conversion of the valuable, or otherwise limiting, target molecule to the desired product. Following the reaction, however, the unreacted functionalized biomolecule or reactive agent must be separated from the labeled target molecule prior to use of the labeled target molecule in a diagnostic assay or as a therapeutic agent. If the unreacted functionalized biomolecule and the labeled target molecule differ sufficiently in size or biophysical properties, chromatographic separation strategies, in particular size-exclusion separation approaches, can be utilized, but such approaches require additional time and materials and often result in a decreased yield of the desired labeled product. If the functionalized biomolecule and the labeled target molecule do not differ sufficiently in size or other biophysical property, such approaches are not helpful.

U.S. Pat. Nos. 6,709,596 B1 and 6,998,041 B2 describe devices and systems for the reversible trapping and isolation of soluble carbohydrates. In these approaches, the trapping device covalently binds activated glycosyl residues, in a reversible manner, to a solid polymeric material that contains aldehydic functional groups. The trapping devices are designed to allow subsequent release of the bound carbohydrates, so that the released material can be further analyzed.

Despite the usefulness of the above approaches, there continues to be a need for the development of improved methods, compositions, and kits for the fast and efficient trapping of biomolecules, in particular biomolecules that have been modified with a reactive group.

### SUMMARY OF THE INVENTION

The present disclosure addresses these and other needs by providing in one aspect methods for trapping a biomolecule. Specifically, these methods comprise, in some embodiments, the step of contacting a solution comprising a functionalized biomolecule with a trapping material, wherein the functionalized biomolecule comprises a reactive amino group, and wherein the trapping material comprises a reactive carbonyl group. In some embodiments, the solution further comprises a labeled target biomolecule, and more specifically, the target molecule can be conjugated with the functionalized biomolecule.

In some embodiments, the functionalized biomolecule is a functionalized oligonucleotide, polypeptide, or polysaccharide, the reactive amino group is a hydrazino group or an oxyimino group, and/or the reactive amino group reacts with the reactive carbonyl group to form a covalent linkage, more specifically a hydrazone or an oxime. In some embodiments, the reactive carbonyl group is a reactive aldehyde group. In some embodiments, the trapping material is a porous resin, a size-exclusion resin, and/or an oxidized polysaccharide resin. In preferred embodiments, the trapping material is prepared by the oxidation of a polysaccharide resin.

In some embodiments, the methods further comprise the step of separating the solution from the trapping material, more specifically wherein the separating step comprises gravity flow separation, magnetic separation, or centrifugation.

In some embodiments, the methods further comprise the step of reacting the functionalized biomolecule with a target molecule prior to the step of contacting the solution comprising the functionalized biomolecule with the trapping material. In these methods, the target molecule can comprise a reactive carbonyl group and/or the methods can further comprise the step of separating the solution from the trapping material, for example wherein the separating step comprises gravity flow separation, magnetic separation, or centrifugation.

In another aspect, the disclosure provides compositions for trapping a biomolecule. More specifically, the compositions can, in embodiments, comprise a functionalized biomolecule and a trapping material, wherein the functionalized biomolecule comprises a reactive amino group, and wherein the trapping material comprises a reactive carbonyl group. In specific embodiments, the functionalized biomolecule can be a functionalized oligonucleotide, polypeptide, or polysaccharide, the reactive amino group can be a hydrazino group or an oxyimino group, and/or the reactive amino group can react with the reactive carbonyl group to form a covalent linkage, such as a hydrazone or an oxime. In other specific embodiments, the reactive carbonyl group can be a reactive aldehyde group. In still other specific embodiments, the trapping material is a porous resin, a size-exclusion resin, and/or an oxidized polysaccharide resin. In some specific embodiments, the trapping material is prepared by the oxidation of a polysaccharide resin.

In some embodiments, the compositions further comprise a labeled target biomolecule. More specifically, the target molecule can be conjugated with the functionalized biomolecule and/or the target molecule can comprise a reactive carbonyl group.

In yet another aspect, the disclosure provides kits for trapping a biomolecule and instructions for using the material to trap a functionalized biomolecule, wherein the trapping material comprises a reactive carbonyl group. In embodiments these kits further comprise a functionalization reagent, for example wherein the functionalization reagent is reacted with a reactive biomolecule to generate a functionalized biomolecule, or further comprise a functionalized biomolecule.

In embodiments of any of the disclosed kits, the functionalized biomolecule can be a functionalized oligonucleotide, polypeptide, or polysaccharide. In other embodiments of any of the disclosed kits, the functionalized biomolecule can comprise a reactive amino group, specifically a hydrazino group or an oxyimino group and/or specifically wherein the reactive amino group of the functionalized biomolecule can be capable of reacting with the reactive carbonyl group of the trapping material to form a covalent linkage, such as a hydrazone or an oxime linkage.

In some embodiments of any of the disclosed kits, the reactive carbonyl group of the trapping material is a reactive aldehyde group. In some embodiments, the trapping material is a porous resin, a size-exclusion resin, and/or an oxidized polysaccharide resin. In some embodiments, the trapping material is prepared by the oxidation of a polysaccharide resin.

### BRIEF DESCRIPTION OF THE DRAWINGS

FIG. 1: Exemplary conjugation reactions showing the incorporation of an amino oxyamino (AOA) moiety into a peptide (top) or an oligonucleotide (bottom) during solid phase synthesis of the peptide or oligonucleotide. The reactions make use of an amino-reactive or hydroxyl-reactive bifunctional conjugation reagent comprising a protected AOA moiety.

FIG. 2: Schematic illustration of the oxidation of a polysaccharide-containing trapping material to generate a reactive carbonyl group (top). Schematic illustration of the reaction of a biomolecule (labeled “polymer”) with a reactive carbonyl of the trapping material. The biomolecule comprises either a hydrazino (R═N) or an oxyamino (R═O) group.

FIG. 3: UV-visible spectra of samples obtained using the trapping compositions and methods of the instant disclosure.

### DETAILED DESCRIPTION OF THE INVENTION

### Methods for Trapping Functionalized Biomolecules

The instant disclosure provides in one aspect methods for the rapid and efficient trapping of functionalized biomolecules, in particular biomolecules that contain a reactive moiety, such as a reactive amino group, or biomolecules that have been labeled with such moieties. Such methods can thereby provide for the rapid and efficient separation of the functionalized biomolecule from a solution that contains other components, for example a target biomolecule, and in particular solutions where the target molecule has been labeled by the functionalized biomolecule.

In the instant methods for trapping, a solution comprising a functionalized biomolecule is contacted with a trapping material, such that the functionalized biomolecule is irreversibly, or nearly irreversibly, associated with the trapping material. Where the solution also comprises a target molecule that has been labeled by the functionalized biomolecule, the excess functionalized biomolecule is thereby separated from the labeled target molecule, because the labeled target molecule does not associate significantly with the trapping material.

The instant methods take advantage of the facile reaction between reactive amino groups and reactive carbonyl groups. As is understood by those of ordinary skill in the art, and as will be described in more detail below, the reaction between these moieties can readily occur in aqueous solution to form a covalent conjugate, for example an imine-based conjugate. Where the reactive amino group is an aliphatic or aromatic amine, the resulting imine-based conjugate is commonly known as a Schiff base.

As noted above, the functionalized biomolecule of the instant methods preferably comprises a reactive amino group. As further noted in the above-cited references, reactive amino groups are often used to conjugate a functionalized biomolecule to an appropriately modified, or otherwise suitably reactive, target biomolecule. As disclosed herein, the reactivity of the reactive amino group can also be utilized in a trapping reaction with a suitable trapping material to remove the functionalized biomolecule from the solution. Specifically, in the instant methods, the trapping material comprises a carbonyl group capable of rapid, efficient, and stable reaction with the reactive amino group of the functionalized biomolecule.

The reactive amino group of the instant functionalized biomolecules can be any amino group that is susceptible to rapid, efficient, and specific capture by the trapping materials of the instant disclosure. In preferred embodiments, the reactive amino group is, or comprises, a reactive hydrazino group or a reactive oxyimino group. These reactive amino groups can be represented chemically as R—NH—NH₂ and R—O—NH_{2,} respectively, where the “R” group represents any suitable chemical moiety, as described more specifically below. Hydrazino and oxyamino groups are known to react with suitable reactive carbonyl groups to form hydrazones and oximes, respectively.

Exemplary reactive hydrazino groups include aliphatic, aromatic, or heteroaromatic hydrazine, semicarbazide, carbazide, hydrazide, thiosemicarbazide, thiocarbazide, carbonic acid dihydrazine, and hydrazine carboxylate groups, as illustrated, for example, in Scheme 1. Exemplary reactive oxy amino groups are described below.

Specific hydrazino labeling reagents, labeling methods, and uses of the labeled products are disclosed in PCT International Publication Nos. WO 01/70685 A2; WO 02/10431 A2; WO 02/10432 A2; WO 02/57422 A2; WO 2008/140452 A1; WO 2011/100493 A1; ; WO 2012/071428 A2; WO 2013/177046 A1; WO 2016/127149 A2; and WO 2018/017606 A1; ; and U.S.

Patent Application Publication No. 2008/0221343 A1; , each of which is incorporated by reference herein in its entirety.

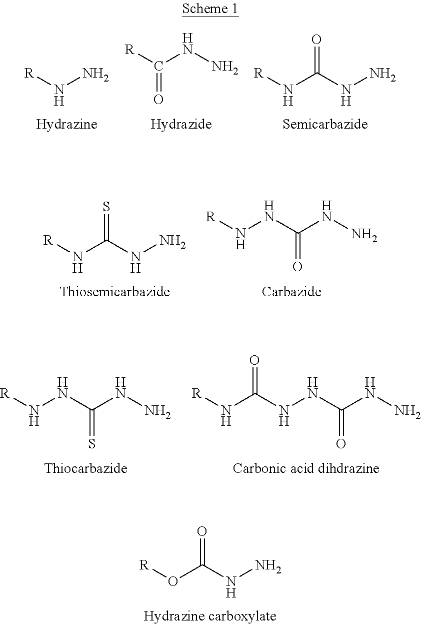

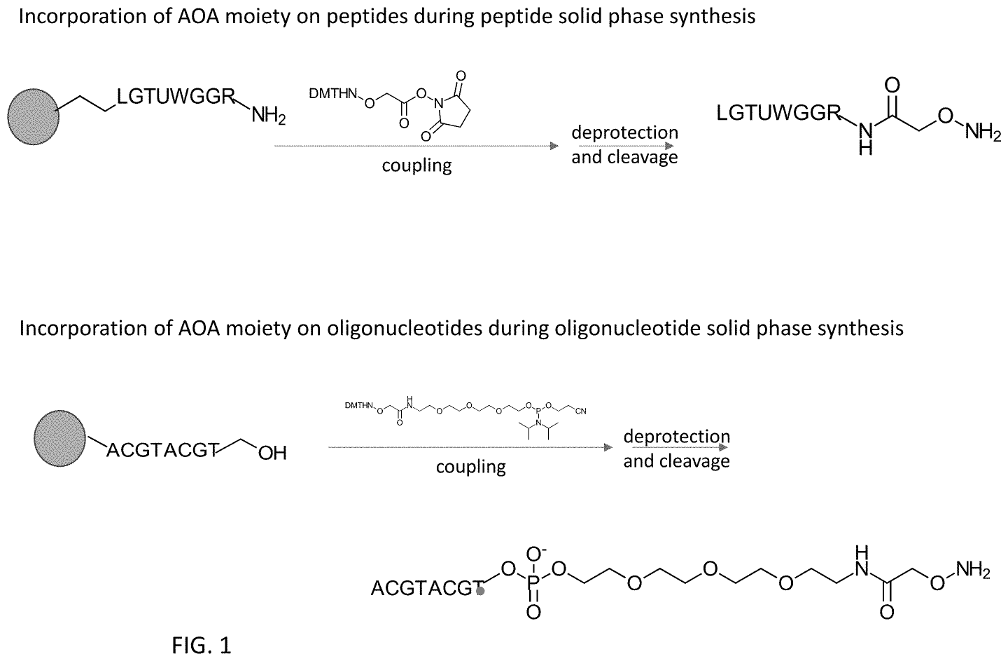

In some embodiments, the “R” group of the above-illustrated hydrazino and oxyamino groups represents a biomolecule that has been functionalized with the corresponding reactive amino group using a suitable coupling reaction or other suitable method, as described in further detail below and in FIG. 1. Although the reactive amino groups can be attached to the biomolecule by any suitable linker, as would be understood by those of ordinary skill in the art, in some embodiments, the reactive amino group is attached to the biomolecule through an aliphatic, aromatic, or heteroaromatic linker. In some embodiments, the reactive amino group is attached to the biomolecule through an amide bond, for example as illustrated in FIG. 1. In some embodiments, the reactive amino group is attached to the biomolecule through a polyethylene glycol (PEG) or another suitable hydrophilic linker group. In some embodiments, the reactive amino group is attached to the biomolecule through a polysaccharide. In some embodiments, the attachment includes more than one of the above-listed linkers, or any other suitable chemical linker, in any combination, as would be understood by those of ordinary skill in the art.

In some embodiments, the reactive amino group of the instant functionalized biomolecules is a protected reactive amino group, such as a protected hydrazino group or a protected oxyamino group. For example, the reactive hydrazino and reactive oxyamino groups can be protected by formation of a salt of the hydrazino or oxyamino group, including but not limited to, mineral acid salts, such as but not limited to hydrochlorides and sulfates, and salts of organic acids, such as but not limited to acetates, lactates, maleates, tartrates, citrates, ascorbates, succinates, butyrates, valerates and fumarates, or any amino or hydrazino protecting group known to those of skill in the art (see, e.g., Greene et al. (1999) Protective Groups in Organic Synthesis (3rd Ed.) (J. Wiley Sons, Inc.)). Alternatively, the reactive amino group can be protected through the formation of an exchangeable hydrazone or oxime, such as, for example the acetone hydrazone of HyNic (6-HydrazinoNicotinamide), which has the following structure:

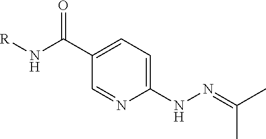

This protected amino group can be used to functionalize a suitable biomolecule or surface (where “R” represents the biomolecule, a surface, or a linker attaching the hydrazone to the biomolecule or surface), for example as described in further detail below. The HyNic acetone hydrazone moiety is readily reactive with aldehydes, in particular aromatic aldehydes, to form stable bioconjugates. The conjugation reaction, which can be catalyzed by the addition of aniline, occurs under mild conditions in aqueous solutions, is highly efficient, and is specific. See Dirksen et al. (2006) J. Am. Chem. Soc. 128:15602.

As described in detail herein, the instant methods for trapping can be used to remove a functionalized biomolecule from an aqueous solution. The functionalized biomolecule of these methods can be any molecule, natural or synthetic. In some embodiments, the functionalized biomolecule is a polymeric molecule. More specifically, the functionalized biomolecule can be a biopolymer, such as, for example, a polypeptide, an oligonucleotide, or a polysaccharide. In specific embodiments, the functionalized biomolecule is a functionalized oligonucleotide or a functionalized polypeptide. Exemplary functionalized biomolecules and their methods of preparation are described in detail in U.S. Pat. No. 7,102,024 B 1, which is incorporated by reference herein in its entirety.

Because the functionalized biomolecules are preferably used in labeling reactions where the target molecules are present at relatively low molar concentrations, and because those target molecules may be expensive and of limited chemical availability, it is highly desirable that formation of the labeled target biomolecule be as efficient and specific as possible and that its formation be complete, or nearly complete, at low molar concentrations of target molecule. It is therefore common practice in such labeling reactions to include an excess of the functionalized biomolecule in order to increase the extent of labeling of the target molecule.

The functionalized biomolecules of the instant disclosure are typically prepared by modification of the biomolecule with a reactive group using a bifunctional conjugation reagent that enables attachment of the reagent to a reactive group on the biomolecule, for example an amino group, a hydroxyl group, or a thiol group. Two exemplary reactions are illustrated in FIG. 1, which shows the reaction of an amino group (top) or a hydroxyl group (bottom) with a suitable bifunctional conjugation reagent. In these examples, the biomolecules themselves have been synthesized using solid-phase synthetic methods, as are well understood by those of ordinary skill in the art. Advantageously, the functional group (in this case a reactive oxyamino group) is added as the last step in the solid-phase synthetic reaction, while the biomolecule remains attached to the synthetic resin in protected form. Only the terminal group (either an amino group for the peptide or a hydroxyl group for the oligonucleotide) is available for reaction with the relevant bifunctional conjugation reagent. Of further advantage in this example, is the ability to remove excess conjugation reagent from the sample by washing the resin after the conjugation reaction has been performed. The functionalized biomolecule, while still typically attached to the solid-phase resin, can then be deprotected and cleaved from the solid-phase resin using known techniques. It should be understood that although the functionalization of a biomolecule can be performed on a solid-phase resin, it can also be performed using solution-phase chemistry, so long as the reaction conditions are suitably specific for generation of the desired product. Bifunctional conjugation reagents, for use in the above conjugation reactions, are available commercially, for example from Solulink, Inc. (San Diego, Calif.) and Jena Bioscience GmbH (Jena, Germany).

The incorporation of hydrazine, oxyamino, and carbonyl-based monomers into biomolecules for use in immobilization and other conjugation reactions is further described in U.S. Pat. Nos. 6,686,461; 7,173,125; and 7,999,098. Hydrazine-based and carbonyl-based bifunctional crosslinking reagents for use in the conjugation and immobilization of biomolecules are described in U.S. Pat. No. 6,800,728. The use of high-efficiency bisaryl-hydrazone linkers to form oligonucleotide conjugates in various detection assays and other applications is described in PCT International Publication No. WO 2012/071428. Each of the above references is hereby incorporated by reference herein in its entirety.

In some embodiments, the functionalized biomolecules of the instant disclosure are prepared using novel functionalization reagents and conditions. For example, a thiol-reactive maleimido oxyamino (MOA) conjugating reagent useful in the preparation of a functionalized biomolecule may be prepared as shown in Scheme 2:

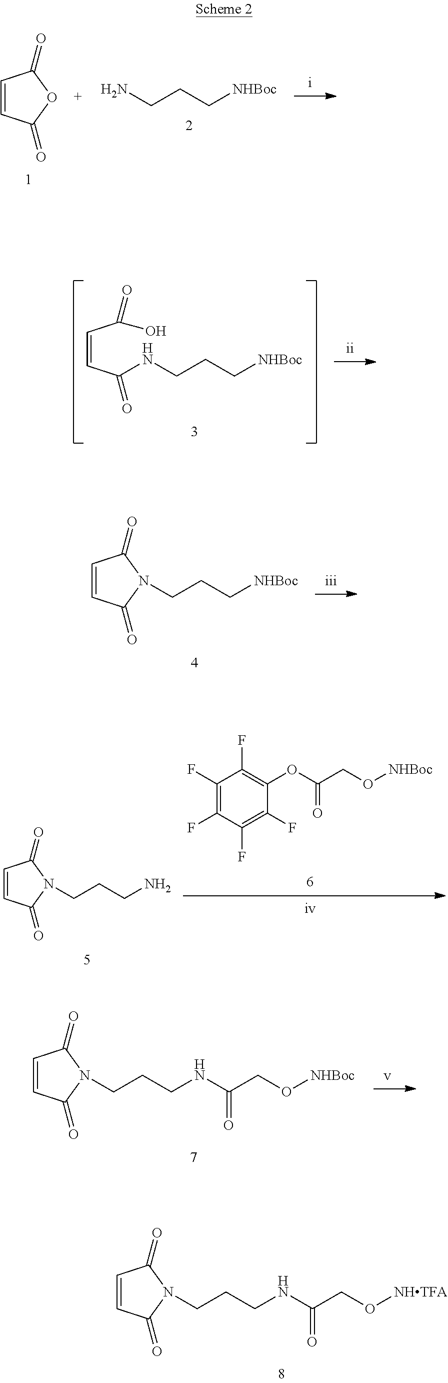

An amino-reactive oxyamino conjugating reagent (AOA) may be prepared as shown in Scheme 3:

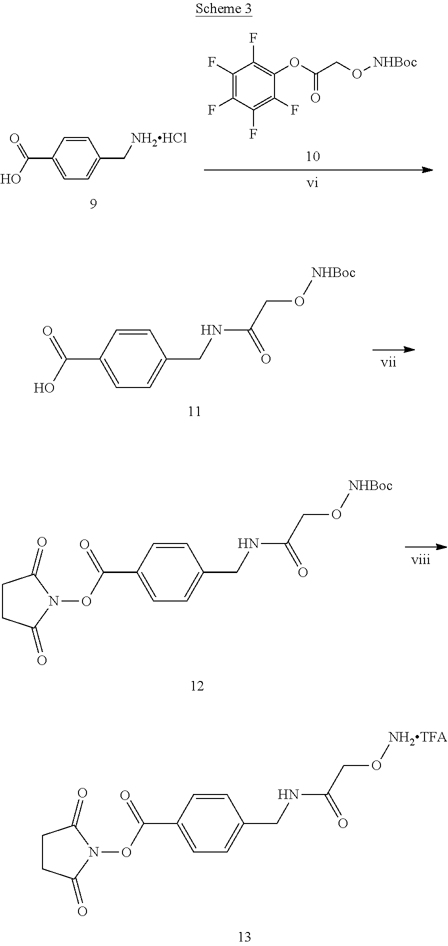

Alternative thiol-reactive, amino-reactive, and hydroxyl-reactive functionalization reagents may be prepared using variants of the above reaction schemes, as would be understood by those of ordinary skill in the art of synthetic chemistry. Such alternative reagents should be considered within the scope of the preparation methods and kits disclosed herein.

Methods of functionalizing a biomolecule with a hydrazino, oxyamino, or other suitable reactive amino group or protected reactive amino group are known in the art, and in particular are disclosed in the above-listed references. Specifically, a bifunctional functionalization reagent, typically with an amino-reactive, thiol-reactive, or hydroxyl-reactive moiety, is reacted with a suitable reactive biomolecule, typically a biomolecule having a reactive amino, thiol, or hydroxyl group at a desired location. It should be noted that when an amino-reactive bifunctional functionalization reagent is used in these reactions, the functionalization agent will typically include a protected form of the reactive amino group, so that the reactivity of the amino group is masked during the functionalization reaction. After the desired biomolecule is functionalized, the protecting group can be removed to allow the conjugation reaction to proceed with an appropriate aldehyde or other suitable conjugation target.

Functionalized biomolecules prepared using one or another of the above oxyamino-containing functionalization reagents may usefully be reacted with a target biomolecule that has itself typically been modified with a carbonyl-containing reagent, for example, an aromatic aldehyde such as a formylbenzoate group. Alternative examples of such a conjugation reactions are shown in Schemes 4 and 5, where the R₁ and R₂ groups represent independently a functionalized biomolecule and a target biomolecule.

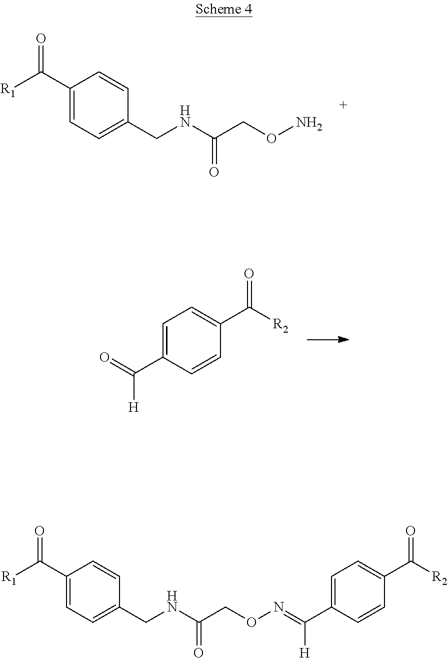

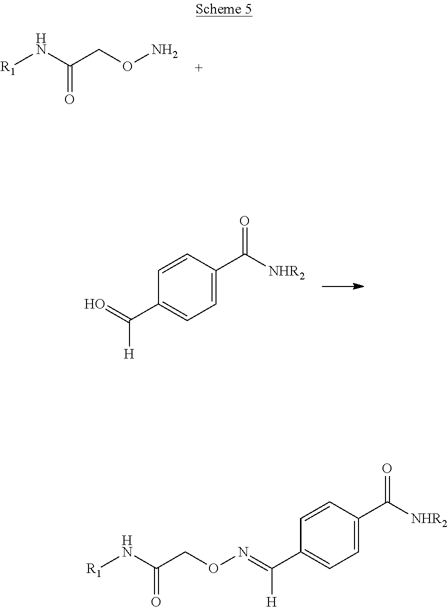

It should be understood that for purposes of the instant trapping methods, it is most typical that the R₁ groups in Schemes 4 and 5 correspond to the functionalized biomolecule, and the R₂ groups correspond to the target molecule. The product in each case represents a target molecule (R₂) that is conjugated with the functionalized biomolecule (R₁). The carbonyl moiety may be any carbonyl-containing group capable of forming a hydrazine or oxime linkage with one or more of the above-described hydrazine or oxyamino moieties. Preferred carbonyl moieties include aldehydes and ketones, and in particular, reactive aldehyde moieties.

The synthesis and stabilities of hydrazone-linked adriamycin/monoclonal antibody conjugates are described in Kaneko et al. (1991) Bioconj. Chem. 2:133-41. The synthesis and protein-modifying properties of a series of aromatic hydrazides, hydrazines, and thiosemicarbazides are described in U.S. Pat. Nos. 5,206,370; 5,420,285; and 5,753,520. The generation of conjugationally-extended hydrazine compounds and fluorescent hydrazine compounds is described in U.S. Pat. No. 8,541,555.

As previously mentioned, the instant methods comprise the step of contacting a solution comprising a functionalized biomolecule with a trapping material, wherein the functionalized biomolecule comprises a reactive amino group, and wherein the trapping material comprises a reactive carbonyl group. In specific embodiments, the trapping material comprises a reactive ketone group or a reactive aldehyde, and more specifically, the reactive carbonyl group is a reactive aldehyde group. Accordingly, in the instant methods, the reactive carbonyl group of the trapping material is capable of reacting with the functionalized biomolecule, ideally in a highly efficient and highly specific manner, so that the trapping material rapidly and efficiently removes most, if not all, of the functionalized biomolecule from the solution. In specific embodiments, the reactive carbonyl group is any carbonyl group capable of forming an imine linkage, for example a hydrazone or oxime linkage, with any of the above reactive amino groups.

In some embodiments, the solutions of the instant methods further comprise an aniline component to catalyze the formation of a hydrazone or oxime conjugate between the reactive amino group and the reactive carbonyl group. See, e.g., Dirksen et al. (2006) Angew. Chem. 45:7581-7584 (DOI: 10.1002/anie.200602877).

In some embodiments, the trapping material is a porous resin. More specifically, in some embodiments, the trapping material is a chromatographic resin, such as a size-exclusion resin. In some embodiments, the trapping material is a polysaccharide resin, more specifically an oxidized polysaccharide resin. Such resins can comprise linear, branched, natural, or synthetic polysaccharides, as would be understood by those of ordinary skill in the art.

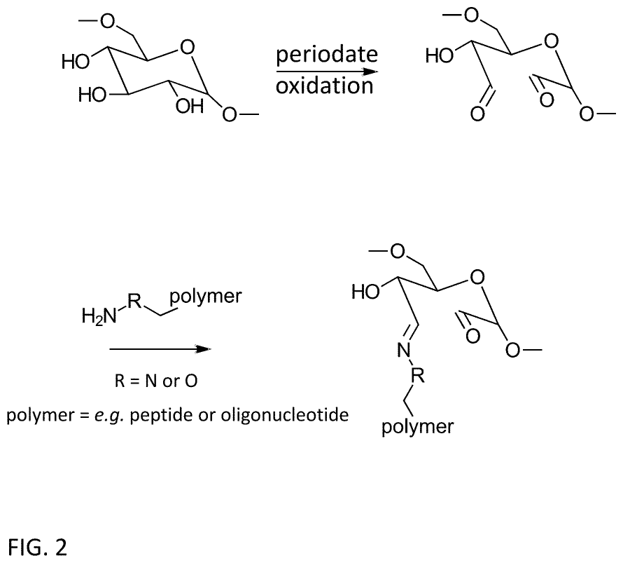

Oxidation of the polysaccharide resin preferably reveals reactive carbonyl groups on the surface of, or within, the resin. Accordingly, in preferred embodiments, the trapping material comprises a reactive carbonyl group, such as a reactive aldehyde group. In some embodiments, the reactive carbonyl group is generated by reaction of the trapping material with a chemical reagent that exposes latent carbonyl groups within the material. For example, polysaccharide resins, such as those described above, are known to react with various oxidation reagents to generate reactive carbonyl groups. Such trapping materials are preferred, as they are inexpensive and relatively easy to prepare. Suitable oxidants useful in the preparation of the instant trapping materials include periodates, such as sodium periodate, and more specifically sodium metaperiodate. An exemplary oxidation reaction is illustrated in FIG. 2 (top). The subsequent reaction of a functionalized biomolecule comprising a reactive amino group is also illustrated in FIG. 2 (bottom), where the reactive amino group is either a hydrazino group (R═N) or an aminooxy group (R═O). The illustrated functionalized biomolecule can be a functionalized peptide or a functionalized oligonucleotide, but it can also be a functionalized polysaccharide or any other suitable functionalized biomolecule.

In other embodiments, the reactive carbonyl group of the trapping material is attached by a coupling reaction, for example by the reaction of a suitable trapping material, for example a hydroxyl-, thiol-, or amino-containing trapping material, with a hydroxyl-reactive, a thiol-reactive, or an amino-reactive bifunctional conjugation reagent, such as the bifunctional conjugation reagents described above for the modification of target biomolecules with a reactive carbonyls, for example, aromatic aldehydes such as the formylbenzoate group.

In some embodiments, the trapping material is a solid surface that contains, or has been modified to contain, a suitable reactive carbonyl group. For example the trapping material can be the surface of a test tube, a storage container, a bead, a tubing material, or any other material having a solid surface that can be contacted by the solution comprising the functionalized biomolecule.

In preferred embodiments, the trapping material is an oxidized corn starch resin or dextran, such as, for example, an oxidized Zeba™ size-exclusion chromatographic resin (available from Thermo Scientific). Such resins are readily oxidized by treatment with sodium periodate, as will be exemplified below.

As noted in the above-referenced patent documents, the reaction between a reactive amino group on the functionalized biomolecule and a reactive carbonyl group on either the target molecule or on the trapping material provides several advantages over traditional crosslinking methods. In particular, the reaction to form a hydrazone or an oxime is specific, efficient, and stable. The specificity means that side reactions, such as homoconjugation reactions, do not occur, or occur at extremely low levels. The efficiency means that the reactions run to completion, or near completion, even at low reagent concentrations, thus generating products in, or near, stoichiometric amounts. The stability of the conjugation moieties formed means that the reactions can be used for a wide variety of purposes, including trapping, without concern that the conjugated products will dissociate during use. In some cases, the above conjugation methods provide the further advantage that the progress of the conjugation reaction can be monitored spectroscopically, since in some of the reactions a chromaphore is formed as the reaction occurs. The instant methods for trapping of the functionalized biomolecule provide still further benefits, since an excess of the unreacted functionalized biomolecule can readily be removed from the reaction solution by these methods.

In some embodiments, the instant methods further comprise the step of separating the solution from the trapping material. As would be understood by those of ordinary skill in the art, this further step provides a solution that is substantially free of the functionalized biomolecule, which has reacted irreversibly, or nearly irreversibly, with the trapping material.

### Compositions for Trapping Functionalized Biomolecules

In another aspect of the instant disclosure are provided compositions for the trapping of functionalized biomolecules. Such compositions comprise a trapping material, as described above, wherein the trapping material comprises a reactive carbonyl group. The compositions further comprise a functionalized biomolecule, for example as described above, wherein the functionalized biomolecule comprises a reactive amino group. It would be understood by those of ordinary skill in the art that such compositions would result from the contacting of a solution comprising the functionalized biomolecule with the trapping material, as is outlined in the above trapping methods.

In specific composition embodiments, the reactive carbonyl group of the trapping material is a reactive aldehyde group.

In other specific composition embodiments, the solution further comprises a labeled target biomolecule. More specifically, the labeled target molecule may be conjugated with the functionalized biomolecule.

In some composition embodiments, the functionalized biomolecule is a functionalized oligonucleotide, polypeptide, or polysaccharide. In some embodiments, the reactive amino group is a hydrazino group or an oxyimino group. In some embodiments, the reactive amino group reacts with the reactive carbonyl group to form a covalent linkage, such as a hydrazone or an oxime.

In some composition embodiments, the trapping material is a porous resin. For example, in some embodiments the trapping material is a size-exclusion resin, is an oxidized polysaccharide resin, or is prepared by the oxidation of a polysaccharide resin. In some embodiments the trapping material is a bead or other solid surface modified to incorporate a reactive carbonyl moiety. In some embodiments, the trapping material is a dextran.

### Kits for Trapping Functionalized Biomolecules

In yet another aspect, the instant disclosure provides kits for use in trapping functionalized biomolecules. In some embodiments, the kits comprise a trapping material, wherein the trapping material comprises a reactive carbonyl group, and instructions for using the material to trap a functionalized biomolecule. Specifically, the instructions provide a user with a description of how to use the trapping material in the above-described methods for trapping a functionalized biomolecule, in particular a functionalized biomolecule comprising a reactive amino group.

In some embodiments, the kits further comprise one or more functionalization reagents. In specific embodiments, the functionalization reagent of the provided kits is reacted with a suitably reactive biomolecule to generate the functionalized biomolecule, for example as described in any of the methods for preparing a functionalized molecule that are described above.

In alternative embodiments, the kits further comprise one or more functionalized biomolecules, for example a functionalized biomolecule prepared according to any of the above methods.

In some embodiments, the biomolecule functionalized by reaction with the kit-provided functionalization reagent, or provided in the kit itself, is an oligonucleotide, a polypeptide, or a polysaccharide.

In more specific embodiments, the functionalized biomolecule of any of the above kits, or prepared using the functionalization reagent of any of the above kits, comprises a reactive amino group. Preferably, the reactive amino group is a hydrazino group or an oxyimino group. In some embodiments, the reactive amino group is capable of reacting with the reactive carbonyl group of the trapping material to form a covalent linkage. More specifically, the covalent linkage is a hydrazone or an oxime.

The trapping material of the provided kits, which comprise a reactive carbonyl group, can be any of the above-described trapping materials. In specific embodiments, the reactive carbonyl group of the trapping material in the provided kits is a reactive aldehyde group.

In some kit embodiments, the trapping material is a porous resin, is a size-exclusion resin, is an oxidized polysaccharide resin, or is prepared by the oxidation of a polysaccharide resin. In some embodiments the trapping material is a bead or other surface modified to incorporate a carbonyl moiety. In some embodiments, the trapping material is a dextran.

In further embodiments, the kits may comprise further components such as, for example, buffers of various compositions to enable usage of the kit for trapping a functionalized biomolecule. Kits may be provided in various formats and may include some or all of the above listed components, or may include additional components not listed here.

It will be readily apparent to one of ordinary skill in the relevant arts that other suitable modifications and adaptations to the methods, compositions, and kits described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Preparation of Oxidized Packing Material

Three 10 mL 40K MWCO Zeba columns (ThermoFisher, Carlsbad, Calif.) were emptied into a 100 mL sintered glass funnel, the supernatant was removed by vacuum and the packing was washed with deionized water (150 mL). To the packing was added DI water (20 mL) and the suspension was transferred to a 50 mL conical flask. A solution of sodium metaperiodate (154 mg) in water (10 mL) was prepared and added to the packing. The mixture was placed on a rotator for 2 h. The packing was transferred to a 100 mL sintered glass funnel and the supernatant removed by vacuum filtration. The packing was washed with DI water (4×50 mL) then PBS/0.05% sodium azide. The packing was resuspended in PBS/0.05% azide (15 mL) and transferred to a 50 mL conical flask-total volume (40 mL). The suspension was aliquoted into Econospin Filters (600 μL; Epoch Lifesciences (Missouri City, Tex.) via pipette.

### Aminooxy-peptide Capture Demonstration

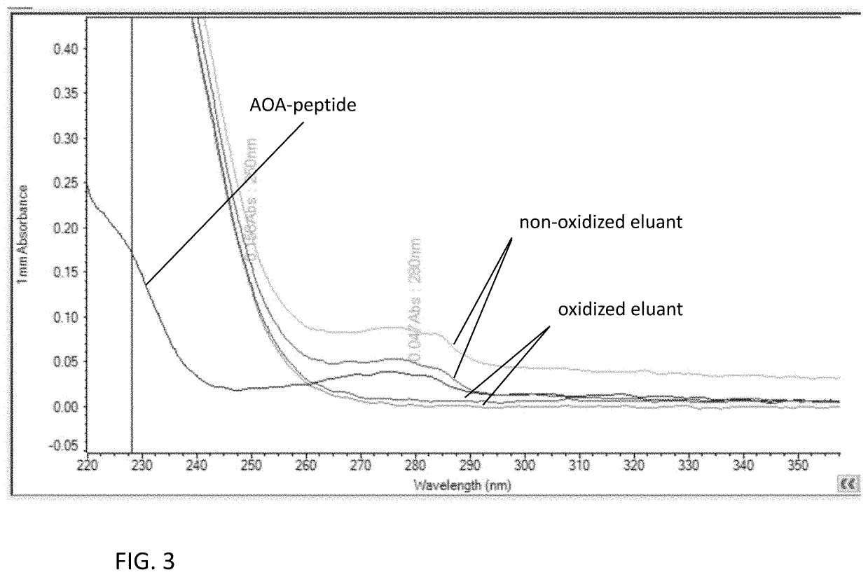

N-terminus-aminooxy-modified 8 mer peptide (“AOA-peptide”) (12.5 μg) was dissolved in 100 mM phosphate, 150 mM sodium chloride, pH 6.0 (50 μL) and placed on an oxidized Zeba packing column prepared as above or on a non-oxidized column. The columns were centrifuged on an Eppendorf microcentrifuge at 1500 rpg for 3 minutes. The UV-visible spectra of the eluants were obtained as shown in FIG. 3. The UV-visible of the AOA-peptide is shown for comparison.

### Synthesis of Conjugate

Antibody (50 μg) in 100 mM phosphate, 150 mM NaCl, pH 7.8 (50 μL) was added to a tube containing sulfo-succinimidyl-4-formylbenzaldehyde (12.5 μg) and incubated for 1 h at room temperature. The reaction mixture was purified and buffer exchanged by filtration through a 0.5 mL spin column with Zeba packing pre-equilibrated with 100 mM phosphate, 150 mM NaCl, pH 6.0. The flow through was transferred to a tube containing the AOA-modified peptide (12.5 μg) and vortexed. To the reaction mixture was added a solution of 100 mM aniline in 100 mM phosphate, 150 mL NaCl, pH 6.0 (5 μL). The reaction mixture was incubated at room temperature for 1 hour.

### Purification of Conjugate

The above conjugation reaction mixture was added to a spin column containing oxidized Zeba packing. The column was centrifuged at 1500 rpg for 2-3 minutes to isolate the purified AOA-peptide-4FB-antibody conjugate.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

1. A method for trapping a biomolecule, comprising the step of:
contacting a solution comprising a functionalized biomolecule with a trapping material;
wherein the functionalized biomolecule comprises a reactive amino group; and
wherein the trapping material comprises a reactive carbonyl group.

2. The method of claim 1, wherein the solution further comprises a labeled target biomolecule.

3. The method of claim 2, wherein the target molecule is conjugated with the functionalized biomolecule.

4. The method of claim 1, wherein the functionalized biomolecule is a functionalized oligonucleotide, polypeptide, or polysaccharide.

5. The method of claim 1, wherein the reactive amino group is a hydrazino group or an oxyimino group.

6. The method of claim 1, wherein the reactive amino group reacts with the reactive carbonyl group to form a covalent linkage.

7. The method of claim 6, wherein the covalent linkage is a hydrazone or an oxime.

8. The method of claim 1, wherein the reactive carbonyl group is a reactive aldehyde group.

9. The method of claim 1, wherein the trapping material is a porous resin.

10. The method of claim 1, wherein the trapping material is a size-exclusion resin.

11. The method of claim 1, wherein the trapping material is an oxidized polysaccharide resin.

12. The method of claim 1, wherein the trapping material is prepared by the oxidation of a polysaccharide resin.

13. The method of claim 1, wherein the trapping material is a solid surface.

14. The method of claim 1, further comprising the step of separating the solution from the trapping material.

15. The method of claim 14, wherein the separating step comprises gravity flow separation, magnetic separation, or centrifugation.

16. The method of claim 15, wherein the separating step comprises centrifugation.

17. The method of claim 1, further comprising the step of reacting the functionalized biomolecule with a target molecule prior to contacting the solution comprising the functionalized biomolecule with the trapping material.

18. The method of claim 17, wherein the target molecule comprises a reactive carbonyl group.

19. The method of claim 17, further comprising the step of separating the solution from the trapping material.

20. The method of claim 19, wherein the separating step comprises gravity flow separation, magnetic separation, or centrifugation.

21. The method of claim 20, wherein the separating step comprises centrifugation.

22.-50. (canceled)
